(19) United States
(12) Patent Application Publication
(10) Pub. No.: US 2021/0375490 A1
(43) Pub. Date: Dec. 2, 2021

(54) Systems and Methods for Auto-Validation of Medical Codes

(71) Applicant: 3M INNOVATIVE PROPERTIES COMPANY, St. Paul, MN (US)

(72) Inventors: Marcus T. Brown, Draper, UT (US); Robert B. Yeganeh, Cottonwood Heights, UT (US)

(21) Appl. No.: 17/309,168

(22) PCT Filed: Oct. 30, 2019
(86) PCT No.: PCT/IB2019/059298

§ 371 Date: May 3, 2021

Related U.S. Application Data
(60) Provisional application No. 62/767,253, filed on Nov. 14, 2018.

(51) Int. Cl. G16H 80/00; G06Q 20/14; G16H 10/60

(52) U.S. Cl. CPC G16H 80/00; G16H 10/60; G06Q 20/14

(57) ABSTRACT

Disclosed are systems and methods for processing medical data. The systems and methods may include processing medical data via one or more computers. The method, executed via the systems may include receiving patient encounter data describing a patient encounter with a healthcare organization. In addition, one or more user defined rules may be received. Once the patient encounter data is received, one or more provider codes may be extracted from the patient encounter data. The method may also include determining that the one or more provider codes satisfies the one or more user defined rules. When the one or more provider codes satisfies the one or more user defined rules, the one or more provider codes may be forwarded to an automated billing system for subsequent billing-related processing.

### TECHNICAL FIELD

This disclosure relates to medical billing for facility and professional services.

### BACKGROUND

In the medical field, accurate processing of patient visits to hospitals and clinics for reimbursement by insurers or other medical payers is important to ensure that medical professionals and facilities receive payment in a timely manner. The emergence of integrated health systems where doctors are employed directly by the hospitals and clinics presents challenges for integrating the processing of patient visits for reimbursement of both facility and physician services.

### SUMMARY

This disclosure describes systems and techniques for processing medical data via one or more computers. The techniques and systems described herein can help to automate (or partially automate) a process of submitting codes for billing. In this manner, the process can be improved and/or simplified. Further, the described techniques and systems may integrate one or more steps taken by a medical reviewer (sometimes referred to as a “documentation specialist”), which may further improve and/or simplify the process for reviewing and adjusting the assignment of reimbursement facts to patient encounter data.

In one example, this disclosure describes a method of processing medical data via one or more computers. The method may include receiving, at the one or more computers, patient encounter data describing a patient encounter with a healthcare organization. In addition, one or more user defined rules may be received at the one or more computers. Once the patient encounter data is received, the one or more computers may extract one or more provider codes from the patient encounter data. The method may also include determining, by the one or more computers, that the one or more provider codes satisfies the one or more user defined rules. When the one or more provider codes satisfies the one or more user defined rules, the one or more computers may forward the one or more provider codes to an automated billing system for subsequent billing-related processing.

In another example, this disclosure describes a computerized medical system for processing medical data. The system may comprise a processor and a memory. The memory may include instructions that, when executed by the processor, cause the processor to perform actions. The actions may comprise receiving patient encounter data describing a patient encounter with a healthcare organization. The actions may also include receiving one or more user defined rules and extracting one or more provider codes from the patient encounter data. The actions may also include determining that the one or more provider codes satisfies the one or more user defined rules and forwarding the one or more provider codes to an automated billing system when the one or more provider codes satisfies the one or more user defined rules for subsequent billing-related processing.

In another example, this disclosure describes a device for processing medical data. The device may comprise means for receiving patient encounter data describing a patient encounter with a healthcare organization. The device may also include means for receiving one or more user defined rules and means for extracting one or more provider codes from the patient encounter data. Means for determining that the one or more provider codes satisfies the one or more user defined rules and means for forwarding the one or more provider codes to an automated billing system when the one or more provider codes satisfies the one or more user defined rules for subsequent billing-related processing may also be included in the device.

In another example, this disclosure describes at least one computer readable storage medium that may include instructions. The instructions, when executed by at least one processor, may cause the at least one processor to process medical data by performing actions. The actions may comprise receiving patient encounter data describing a patient encounter with a healthcare organization and receiving one or more user defined rules. The actions may also include extracting one or more provider codes from the patient encounter data and determining that the one or more provider codes satisfies the one or more user defined rules. The actions may also include forwarding the one or more provider codes to an automated billing system when the one or more provider codes satisfies the one or more user defined rules for subsequent billing-related processing.

In some instances, provider codes extracted from patient encounter data may be sent directly to a billing system without review by a medical reviewer. In other instances, the provider codes extracted may be selected for review by a medical reviewer before being sent to a billing system. Whether provider codes are sent to the billing system with or without being sent to a medical reviewer may be based on rules that a user, such as a system administrator or delegated individuals, has entered into the systems disclosed herein.

The techniques and systems described herein can help to simplify the billing process by allowing selected provider codes extracted from patient encounter data to be sent directly to a billing system without medical reviewer intervention. The techniques may operate to reduce the number of documentation specialists needed to review provider codes before billing can take place. The techniques may further describe methods and systems for simplifying and streamlining the process of having provider codes sent to billing systems, thereby further reducing the overhead and time between when products and services are provided and insurers or other payment providers are billed.

As described in greater detail below, the methods of this disclosure may be performed by one or more computers. The methods may be performed by a standalone computer, or may be executed in a client-server environment in which a documentation specialist views the patient encounter data at a client computer, an administrator enters rules, etc. In the latter case, the client computer may communicate with a server computer. The server computer may store the patient encounter data, provider codes, user defined rules, user codes, etc. and apply the techniques of this disclosure to facilitate review of the patient encounter data when necessary at the client computer and forwarding the provider codes to a billing system.

### BRIEF DESCRIPTION OF THE FIGURES

FIG. 1 shows a block diagram illustrating an example of a stand along computer system for processing medical data consistent with this disclosure.

FIG. 2 shows a diagram illustrating an example user interface window for enter user defined rules consistent with this disclosure.

FIG. 3 shows an example report consistent with this disclosure.

FIG. 4 shows a flow diagram illustrating a method consistent with this disclosure.

### DETAILED DESCRIPTION

The following detailed description refers to the accompanying drawings. Wherever possible, the same reference numbers are used in the drawings and the following description to refer to the same or similar elements. While embodiments and examples are described, modifications, adaptations, and other implementations are possible. For example, substitutions, additions, or modifications may be made to the elements and stages illustrated in the drawings, and the systems and methods described herein may be modified by substituting, reordering, or adding stages to the disclosed methods or elements to the disclosed systems. Accordingly, the following detailed description does not limit this disclosure. Instead, the proper scope of any inventions disclosed herein is defined by the appended claims.

The systems and methods disclosed herein may allow for the ability to automate the review of medical coding and billing with no user intervention using Natural Language Processing (NPL), physicians coding and reimbursement systems (PCRS) or coding and reimbursement system (CRS). As disclosed herein, NLP may validate the codes chosen, while the coding system identifies invalid coding combinations and provides all the additional information required on the billing form.

As disclosed herein, the validation of provider supplied code used in medical claims submission may be automated by allowing a user to program a code or group of codes that can be used to validate provider supplied codes. The provider supplied codes can be reviewed to validate that they meet specific clinical and regulatory requirements. User defined rules based on multiple criteria to apply validations to claims with specific criteria may be created. Claims or medical codes that do not pass a rule, or validation criteria, may be held for review and manual coding as necessary. In addition, statistical data regarding the performance of auto-validation, claims or medical codes that did not pass validation and why, and statistical sampling of claims that pass validation for review by a human may be provided.

The systems and methods disclosed herein may allow claims to be analyzed and determine which claims have highest risk of error based on criteria defined in user supplied rules. If the claim passes the rule and meets the criteria, it may be sent directly to the billing system. The rules may be built to identify criteria a user wants to set for determining risk. Rules can be based on multiple criteria and include, but are not limited to, medical provider, facility location, medical specialty, specific code or codes, code pairs, coding levels, code category, regulatory and clinical requirements. For claims that fail to satisfy a rule, manual coding of the medical claim may be provided. The rules may also include regulatory and clinical requirements so as to reduce the frequency that a claim will be rejected. Sending claims or medical codes to a billing system may include completing data required for the claim to be accepted. Examples of this data includes, but is not limited to, provider name, billing codes, location type, etc.

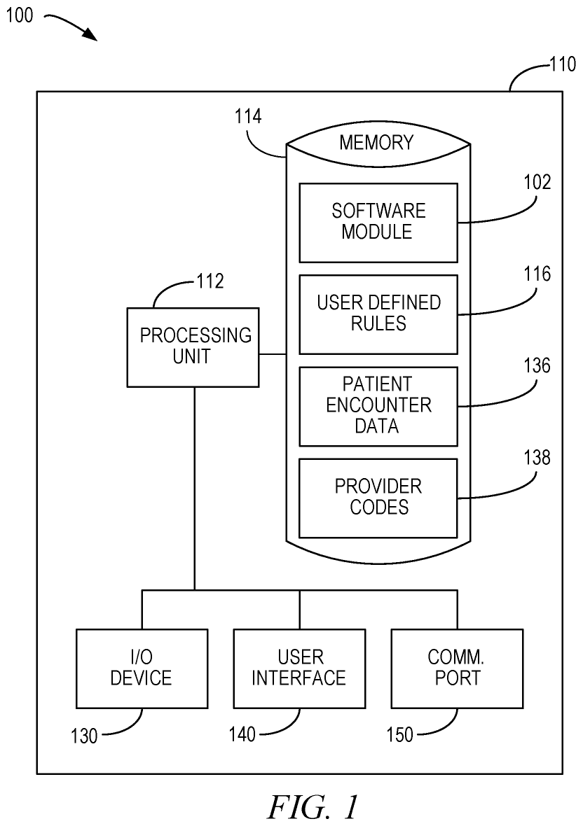

FIG. 1 is a block diagram illustrating an example of a stand-alone computerized system 100 for transmitting provider codes and/or patient encounter data to a billing system consistent with this disclosure. The system 100 comprises a computer 110 that includes a processor 112, a memory 114, an input/output (I/O) device 130, a user interface 140, and a communications port 150. Computer 110 may also include many other components. The illustrated components are shown merely to explain various aspects of this disclosure.

I/O device 130 may comprise a display screen, although this disclosure is not necessarily limited in this respect, and other types of output devices may also be used. Memory 114 stores patient encounter data 118, which may comprise data collected in documents such as patient medical records. Memory 114 may further store provider codes 120 and user defined rules 116. Memory 114 may include a software module 102 that executes techniques of this disclosure.

Processor 112 may comprise a general-purpose microprocessor, a specially designed processor, an application specific integrated circuit, a field programmable gate array, a collection of discrete logic, or any type of processing device capable of executing the techniques described herein. In one example, memory 114 may store program instructions (e.g., software instructions in software module 102) that are executed by processor 112 to carry out the techniques described herein. In other examples, the techniques may be executed by specifically programmed circuitry of processor 112. In these or other ways, processor 112 may be configured to execute the techniques described herein.

I/O device 130 may comprise a display screen, and may also include other types of output capabilities. In some cases, I/O device 130 may generally represent both a display screen (touchscreen or otherwise) and a printer in some cases. Software module 102 may be configured to cause I/O device 130 to output patient encounter data 136 and provider codes 138. Patient encounter data 136 and provider codes 138 may be generated, e.g., as output on a display screen such as user interface 140, so as to allow a documentation specialist to add or modify the provider codes 138 via I/O device 130. For example, patient encounter data 136 and provider codes 138 may be displayed on a display and a documentation specialist may review and/or alter provider codes 138 to be consistent with the patient encounter data 136. The techniques of this disclosure may serve to allow the documentation specialist to verify and edit all provider codes 120 in a single workflow prior to provider codes 136, or billing facts generated via provider codes 136, being transmitted to a billing system.

Communications port 150 may allow computer 110 to communicate with various information sources and devices, such as, but not limited to, remote computing devices, mobile devices, peripheral devices, etc. Non-limiting examples of communications port 150 include, Ethernet cards (wireless or wired), BLUETOOTH® transmitters and receivers, near-field communications modules, etc.

In one example, software module 102 receives patient encounter data 118 and provider codes 120. In some examples, software module 102 may receive patient encounter data 118 in response to an indication to begin processing one or more patient medical records. Patient encounter data 118 may include information included in a patient medical record or any other documents or files describing a patient medical encounter with a healthcare facility. For example, when a patient has an encounter with a healthcare facility, such as during an inpatient admission or an outpatient visit, all of the information gathered during the encounter may be consolidated into a patient medical record. In one example, such a patient medical record may include any procedures performed, any medications prescribed (and given), any notes written by a physician or nurse, and generally any other information concerning the patient encounter with the medical facility. In some examples, the patient medical record may include one or more facility reimbursement facts, one or more professional reimbursement facts, or both.

The facility reimbursement facts and the professional reimbursement facts may be any information related to reimbursement for the services performed and equipment used during the patient medical encounter. These reimbursement facts may include, but are not limited to, medical billing codes. Examples of such medical billing codes include codes associated with the International Classification of Diseases (ICD) codes, Current Procedural Technology (CPT) codes, a Healthcare Common Procedural Coding System codes (HCPCS), and Physician Quality Reporting System (PQRS) codes. In some examples, the codes associated with facility reimbursement include ICD codes, CPT codes, and HCPCS codes. Generally, these reimbursement facts are related to the services and equipment provided by the facility where the patient encounter occurred. Codes associated with professional reimbursement may include ICD codes, CPT codes, and PQRS codes. Generally, these reimbursement facts are related to the services and equipment provided by the attending medical professional. In other examples, the facility and professional reimbursement facts may include any medical billing codes.

As some examples, services provided by the facility may include nursing services or other services performed by employees of the facility. Examples of equipment may include various needles, IV tubing, diagnostic equipment, and other such medical equipment used during the patient encounter. Services provided by the attending medical professional (i.e. a medical doctor) may include diagnosis and treatment. Ultimately, these various reimbursement facts are supposed to describe the patient encounter including a diagnosis or reason for the encounter, a description of the services provided by both the facility and the medical professional, and a description of the equipment used by the facility to assist in diagnosis and treatment of the patient. By providing reimbursement facts to a payer (e.g. a governmental payer, or an insurance company, or any other person or entity paying for medical care), the facility and medical professionals are able to recoup costs and earn revenue based on the actual services provided and equipment used during the patient encounter.

Software module 102 may further determine one or more facility or professional reimbursement facts included based on the patient encounter data 118. In at least one example, various medical professionals or healthcare facility employees may have already entered various reimbursement facts into a patient medical record during or shortly after the patient medical encounter. In such examples, software module 102 may simply identify those previously entered reimbursement facts. In other examples, software module 102 may determine the reimbursement facts based on natural language processing of the patient medical encounter. Generally, the natural language processing entails parsing information, such as that contained in a patient medical record, extracting keywords, and matching those keywords to one or more reimbursement facts. In still other examples, software module 102 may determine reimbursement facts by both identifying previously entered reimbursement facts and by performing natural language processing on the patient medical record.

After determining one or more reimbursement facts based on patient encounter data 118, software module 102 may further utilize user defined rules 116 to determine when claims based on the provider codes or reimbursement facts are to be forwarded to a billing system. For example, user defined rules 116 may include rules that define criteria for automatically submitting provider codes 138 or reimbursement facts derived from provider codes 138 to the billing system. For instance, patient encounter data or provider codes supplied by a given provider, group of providers, or other identified personnel may be known to generally be accurate and thus require less oversight or review before being submitted to the billing system. As an example, an administrator may know that Dr. John Doe or doctors associated with a specialty, such as cardiology, generally enter patient encounter data and provider codes that are correct. As a result, upon processing unit 112 receiving patient encounter data 136 or provider codes 138 from Dr. John Doe or personnel from the cardiology department, who are identified in user defined rules 116, processing unit 112 may transmit a claim based on the provider codes 138 or patient encounter data 118 to the billing system for subsequent billing.

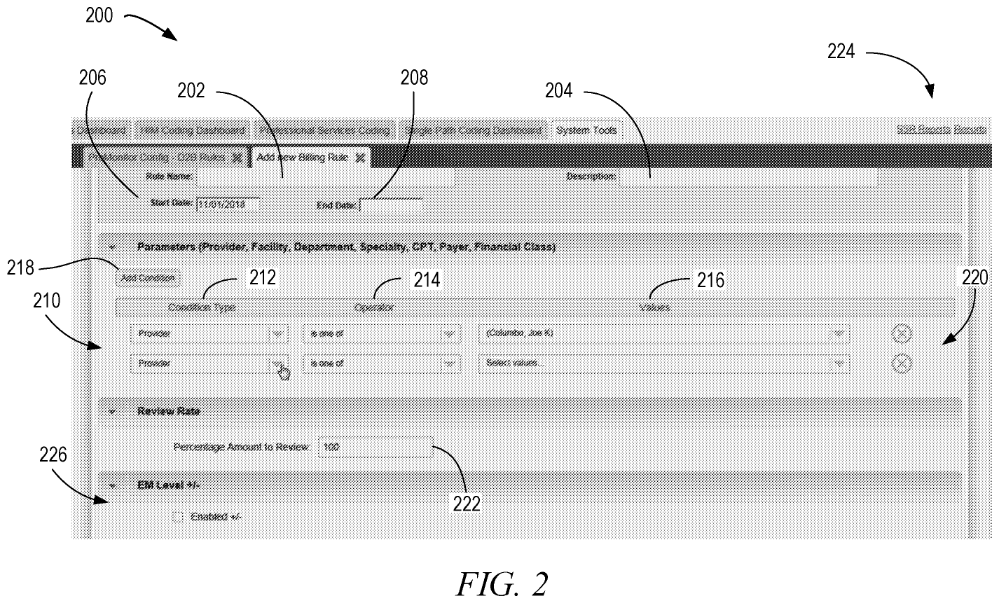

Software module 102 may also output a user interface. The user interface may allow administrators or other personnel to enter user defined rules 116. As disclosed herein, the user interface may include condition types, operators, values, etc. that allow for creation or user defined rules 116. The values may be accessible via dropdown menus or directly entered as disclosed with respect to FIG. 2, below. User interface may include a single or multi-window user interface. Regardless of the number of windows user interface includes, software module 102 may output the user interface to I/O device 130. As described above, I/O device 130 may be a display screen of one or more computers. Some example display screens include a monitor of a desktop computer or a laptop computer. In other examples, the display screen may be connected to a tablet computer or a mobile phone. In other examples, the display screen may not be associated with a single device, but may include the ability to connect to multiple computing devices. Further, I/O device 130 is not limited solely to display screens. For example, I/O device 130 may comprise a computer connected to computer 110, or any other known output device.

Software module 102 may compare provider codes 138 using user defined rules 116 to determine when a claim based on patient encounter data 136 and/or provider codes 138 can be automatically sent to a billing system without review. For example, part of patient encounter data 138 may include the name of a physician. One of the rules within user defined rules may indicate that patient encounter data 136 created by the physician is to be sent directly to billing without review by a medical reviewer. In addition, user defined rules 116 may include a rule that a given percentage of patient encounter data 136 and created by the physician are to be randomly selected for review by the medical reviewer. By selecting random samples for review quality control can be maintained. In addition, personal that may have consistent errors may be noted and additional training or other remedial action may be initiated. In these examples, when software module 102 receives patient encounter data 136 or provider codes 138, software module 102 may transmit the data accordingly.

The system of FIG. 1 is a stand-alone system in which processor 112 that executed software module 102 on the same computer 110. However, the techniques of this disclosure may also be performed in a distributed system that includes a server computer and a client computer. In this case, the client computer may communicate with the server computer via a network and communications port 150. The software module may reside on the server computer, but the output device may reside on the client computer. In this case, when the software module causes display prompts, the software module causes the output device of the client computer to display the prompts, e.g., via commands or instructions communicated based on the server computer to the client computer.

FIG. 2 shows a computer screenshot that may be representative of a user interface 200 according to aspects of this disclosure. For example, the screenshot may be output at I/O device 130 of computer 110 shown in FIG. 1. The screenshot may be generated as part of the coding process (i.e. software module 102) executed by processor 112 of computer 110 shown in FIG. 1.

A user, such as an administrator, may use user interface 200 to input one or more user defined rules. The rules created using user interface 200 may be used by computer 110 to select patient medical records that can be sent directly to a billing system instead of being reviewed by a medical reviewer. For example, when a patient contact satisfies certain conditions, the provider codes, patient encounter data, or other billing information may be sent directly to billing system without having to be reviewed by a medical reviewer.

To create a rule the user may first name the rule by entering a text string in text box 202. The rule name may be descriptive, such as “Dr. John Doe,” for rules that may apply to a give provider. In addition, the rule name may be a generic name such as, “cardiologist” or “echocardiogram” for rules that apply to a given group of people or service.

Using text box 204 the user may enter a description for the rule. When the rule has a generic name, the description may allow the user to enter information so as to describe the nature of the rule or any conditions set forth in the rule. For example, if the rule name is “sonogram” the description may indicate the rule applies to echocardiograms, which are a specific type of sonogram.

User interface 200 may also include a start date box 206 and an end date box 208. By having a start date and an end date, rules can be set to apply during given time periods. For example, there may be a rule that only applies during non-flu season time of the year. The rule may include a set of provider codes that if appearing in patient encounter data 136 may indicate that a person has the flu. Thus, during non-flu season the provider codes may trigger the rule so as to cause provider codes and patient encounter data 128 to be reviewed by a medical reviewer as there may be an error if the data indicates a patient has the flu during non-flu season.

In a parameter section 210 the user may define the rule. Defining the rule may include setting a condition type 212. Condition type 212 may define a condition for the rule. Examples of condition type 212 include provider, service, product, procedure, medication, etc.

An operator 214 may be used to define when a value 216 appears in the condition type 212. Operator 214 may be any Boolean operator. For instance, operator 214 may include, but is not limited to, IS ONE OF, IS NOT ONE OF, AND, OR, XOR, EQUALS, NOT EQUAL, etc.

Value 216 may include a value for provider codes 138 and/or patient encounter data 136. Non-limiting examples of value 216 a service provider's name, a service provider's specialty, a facility where the medical encounter took place, a department where the medical encounter took place, a medication given to a patient, a dosage for the medication, a medication prescribed to the patient, a name of a service provided to the patient, etc.

To create the rule, the user may select an add condition button 218. Once a condition is added, the user may use drop down menus to select condition type 212, operator 214, and value 216. Condition type 212, operator 214, and value 216 may be predefined values stored in a database, such that they are selectable via drop down menus as shown in FIG. 2. Also, condition type 212, operator 214, and value 216 may be text strings that may be enterable via text box.

Rules can also be removed using cancel buttons 220. For example, if a component of a rule is no longer needed, then the component of the rule can be removed. For instance, if Joe Columbo is no longer employed by the service provider, then the portion of the rule that references him can be removed.

In addition to the conditions, other parameters for the rule can be added using user interface 200. As shown in FIG. 2, a review rate can be set text box 222. The review rate can set to any value between 0% and 100%. The review rate may be set so that randomly selected patent encounter data 136 that satisfy the various conditions for the rule can be sent to a medical reviewer. Stated another way, the review rate can be set so that a given percentage of patient encounter data 136 are reviewed for quality control before being sent to a billing system.

User interface 200 may also allow a user to select a range of for codes. For instance, user interface 200 may include a section 226 that allows a user to include a range of plus or minus evaluation and management (EM) levels. The EM levels may allow for a range of codes to be acceptable for a given code. For example, a code may include five digits and the last digit may represent a severity for a condition and the first four digits may represent the condition. Thus, if the code is 12345, 1234 may represent the condition such as a heart attack and 5 may represent the severity of the condition, such as a severe heart attack. Since not all heart attacks are severe, a code of 12341 may also be an acceptable code for a heart attack. Thus, use of various EM levels may allow for suffixes for codes.

Other criteria may also be included with the rule. For example, user interface 200 may include other sections that allow the user to create other actions to be taken when the conditions of the rule are met. For instance, use of a natural language processor can be turned on or off, a time can be set to hold a bill, etc. As an example, a provider may wish to hold all bills for 60 days as part of an agreement with a payor. As a result, the user can set a number of days held until released value to 60.

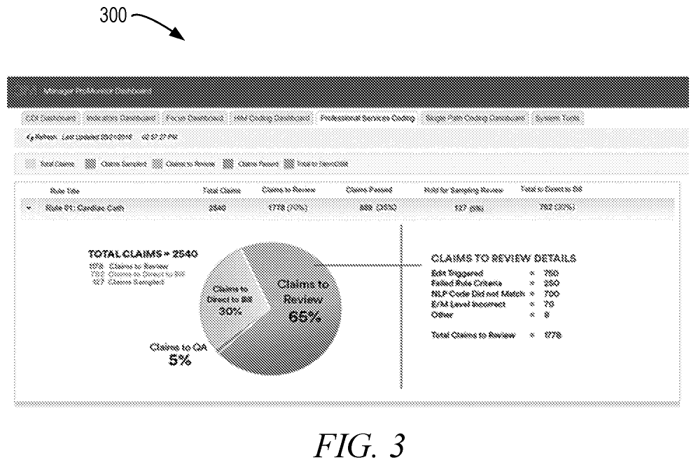

User interface 200 may also include a link or other input 224 that may allow the user to generate reports. The reports may be used to perform analytics on data that satisfies or dissatisfies the rule. For example, software module 102 may include instructions that gather statistics for data. FIG. 3 shows an example of a report 300. As shown in FIG. 3, report 300 can show a total number of claims, a total number of claims to be reviewed, the portion of claims selected for quality control, the total number of claims sent to direct billing, etc. Other parameters can be setup when the system is created or as needed and shown in reports as needed. Once set up using user interface 200, the rules may be stored in a database, such as a SQL database. Having the rules stored in a database may allow the database to persist the rules as needed to various computers.

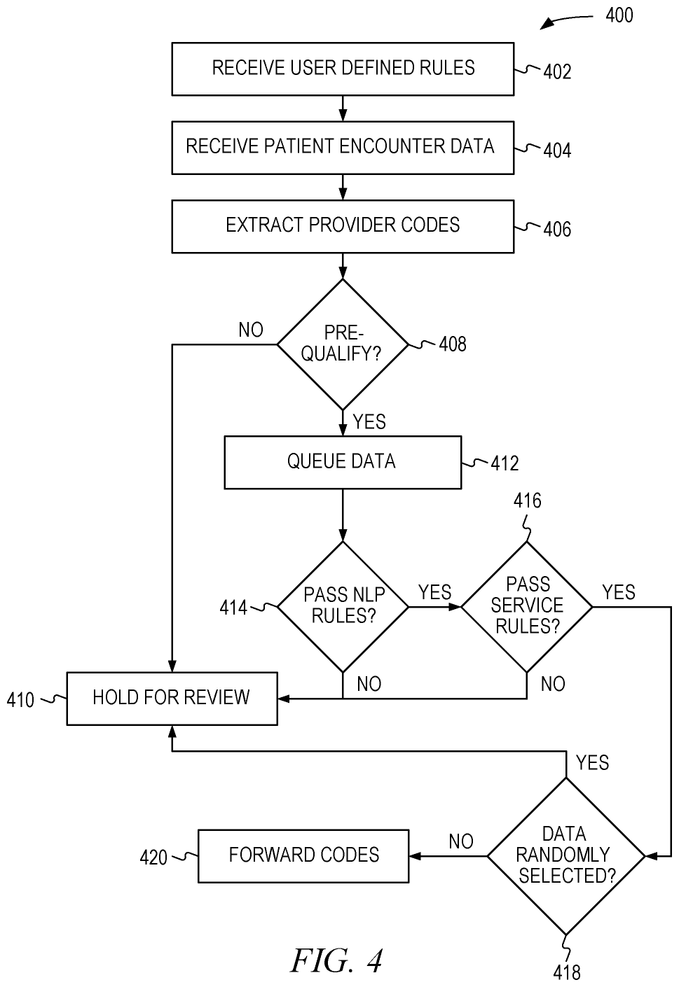

FIG. 4 shows a flow diagram illustrating a method 400 consistent with this disclosure. FIG. 4 will be described from the perspective of computer 110 of FIG. 1, although other computing systems such as distributed systems could also be used to perform method 400. Method 400 may begin at stage 402 where computer 110 may receive user defined rules 116. As disclosed herein, receiving the user defined rules 116 may be inputted using user interface 200. User defined rules 116 may include various conditions that may identify various risks that a claim may be rejected. For example, if provider codes found in the claim fail to satisfy the rules or include inconsistencies, then the claim may be flagged and held for review as disclosed herein.

From stage 402 method 400 may proceed to stage 404 where patient encounter data 136 may be received. As disclosed herein, I/O device 130 or user interface 140 may be used to input patient encounter data 136. Patient encounter data 136 may be received by scanning a document that includes information that can be extracted or a physician or other provider directly entering patient encounter data 136 into computer 110.

From stage 404 method 400 may proceed to stage 406 where provider codes 138 may be extracted from patient encounter data 136. For example, patient encounter data 136 may be received as an XML document and computer 110 may parse the XML document to extract the provider codes that are found within patient encounter data 136 to be used in generating a claim to be submitted to a payor, such as the government or an insurance company.

From stage 406 method 400 may proceed to decision block 408 where a determination may be made as to if the patient encounter data prequalifies for direct billing. The prequalification stage may allow for a cursory filtering of patient encounter data. For example, certain providers or specialties, such a cardiology, may be known to be accurate in entering patient encounter data such that provider codes entered by the providers or specialties are generally correct. Thus, those providers and specialties may qualify for direct billing. Some providers and specialties may not qualify for direct billing. The non-qualifying providers and specialties may be due to poor quality in the past, because of a payor's requirement that codes be verified by a person before billing, etc. The rules for determining if a visit prequalifies may be part of the user defined rules received in stage 402. The rules may also include determine if the patient encounter data is marked as a final document by a provider. Additional rules may include does the patient encounter data originate from a give facility or include specific codes.

When the patient encounter data or the extracted provider codes do not prequalify, method 400 may proceed to stage 410 where the provider codes may be held for review by a medical reviewer. When the visit prequalifies, method 400 may proceed to stage 412 where the data, such as the patient encounter data and provider codes, may be queued for further examination. For instance, the data may be passed to a queue table and remain there for a predefined period of time before being processed. This time delay may give the system enough time to gather the information needed from other programs such as auto coding results. It also gives provider enough time to send any updates for the documents.

From stage 412 method 400 may proceed to decision block 414 where a determination may be made as to whether or not the data passes NLP rules. If the data does not pass NLP rules, method 400 may proceed to stage 410 where the data may be held for review by a medical reviewer. Passing NLP rules may include passing the data, such as the patient encounter data, to a natural language processor that may analyze the data to determine if the extracted provider codes match an expected value. For example, one of the notes in the patient encounter data may be “performed echocardiogram” and the NLP may determine that based on this text string a service code for an echocardiogram should be present in the extracted provider codes. If the code is found, the data passes the NLP rules and if the code is not found the data does not pass the NLP rules.

NLP services may use machine learning to auto suggest codes. For example, patient encounter data and codes that a reviewer assigned for the data may be used to train the NLP to identify codes for similar patient encounter data. As a result, machine learning may be used to provide end users with suggestions when they create the rules.

The NLP services may include analyzing documents, such as patient encounter data, to extract information for a form implemented on a computer system, such as computer 110. For example, the computer system may receive documents of a subject, for example, a patient, a case, and the like. The computer system may identify codes that may correspond to text in the documents. The computer system may first extract evidences from the documents for codes using a natural language processor. The system may then analyze the evidences with a machine learning processor to determine the suggested codes. For example, the suggested codes may be generated based on the most recent evidence. As another example, the suggested codes may be generated based on the supporting evidence. As yet another example, the suggested codes may be generated based on the negating evidence. After the suggested codes are generated, the computer system may export the provider codes. Further description of the NLP services can be found in International Patent Application Number PCT/US2018/028061, the contents of which are hereby incorporated by reference in its entirety.

If the data passes the NLP rules method 400 may proceed to decision block 416 where a determination may be made as to whether the data passes service rules. The service rules may be standard set of rules that apply to everybody and are always check. The service rules may check to see if standard instruct practices are being followed. Service rules may include determining if certain procedures are allowed to be direct billed. For example, certain procedures may require certain diagnostic codes. Thus, if the procedure code is present and the diagnostic code is not present the data may fail the decision block 416 and method 400 may proceed to stage 410 where the data may be held for review by a medical reviewer.

If the procedure is eligible for direct billing, method 400 may proceed to decision block 418 where a determination may be made as to if the data is to be randomly selected for quality review. As detailed herein, a certain percentage of data may be held for quality review. If the data is to be held for quality review, method 400 may proceed to stage 410 where the data may be held for review. If the data is not to be held for quality review, method 400 may proceed to stage 420 where the provider codes may be forwarded to a billing system for direct billing. The billing system may use the provider codes to generate and submit a claim to a payor.

If the claim is randomly selected to be held, then method 400 may proceed to stage 410 where the claim may be held for review by a medical reviewer. If the claim is not randomly selected for review, then method 400 may proceed to stage 420 where the codes may be forwarded to a billing system where the billing system may create a claim using the codes for direct billing.

The various stages and decision blocks do not have to be include in method 400. For example, NLP decision may be turned on or off using user interface 200. As a result, during execution of method 400, method 400 may proceed to decision block 418 from decision block 414. Each of the decision blocks in method 400 may be defined by the user defined rules. Each of the decision blocks may also include more than one rule.

The techniques of this disclosure may be implemented in a wide variety of computer devices, such as servers, laptop computers, desktop computers, notebook computers, tablet computers, hand-held computers, smart phones, and the like. Any components, modules or units have been described provided to emphasize functional aspects and does not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

1. A method of auto-validating medical codes via one or more computers, the method comprising:
receiving, at the one or more computers, patient encounter data describing a patient encounter with a healthcare organization;
receiving, at the one or more computers, one or more user defined rules;
extracting, via the one or more computers, one or more provider codes from the patient encounter data;
determining, via the one or more computers, that the one or more provider codes satisfies the one or more user defined rules; and
forwarding, via the one or more computers, provider codes to an automated billing system when the one or more provider codes satisfies the one or more user defined rules for subsequent billing-related processing.

2. The method of claim 1, wherein receiving the one or more user defined rules further comprise:
generating a user interface including an input section for receiving the one or more user defined rules; and
displaying the user interface at a display device.

3. The method of claim 1, wherein the one or more provider codes include medical billing codes.

4. The method of claim 1, wherein the one or more provider codes identifies a facility reimbursement fact or a professional reimbursement fact.

5. The method of claim 4, wherein the facility reimbursement fact comprises at least one of an International Classification of Diseases (ICD) code, a Current Procedural Technology (CPT) code, or a Healthcare Common Procedural Coding System Code (HCPCS).

6. The method of claim 4, wherein the professional reimbursement fact comprises at least one of an International Classification of Diseases (ICD) code, a Current Procedural Technology (CPT) code, or a Physician Quality Reporting System (PQRS) code.

7. The method of claim 4, wherein the facility reimbursement fact and the professional reimbursement fact include at least one of a provider name, a provider specialty, a product provided, and a service provided.

8. The method of claim 7, further comprising:
selecting, at random, one of the plurality of patient encounter data sets; and
transmitting the one of the plurality of patient encounter data sets to a documentation specialist for verification.

9. The method of claim 8, wherein selecting the one of the plurality of patient encounter data sets includes selecting a predefined percentage of the plurality of patient encounter data sets.

10. The method of claim 1, wherein receiving the patient encounter data includes receiving an image of a document, the document containing the patient encounter data.

11. The method of claim 1, wherein receiving the patient encounter data includes receiving a plurality of patient encounter data sets, each of the plurality of patient encounter data sets corresponding to a respective patient.

12. A computerized medical system for auto-validating medical codes, the system comprising:
a processor; and
a memory comprising instructions that, when executed by the processor, cause the processor to perform actions comprising:
receiving patient encounter data describing a patient encounter with a healthcare organization,
receiving one or more user defined rules,
extracting one or more provider codes from the patient encounter data,
determining that the one or more provider codes satisfies the one or more user defined rules, and
forwarding the one or more provider codes to an automated billing system when the one or more provider codes satisfies the one or more user defined rules for subsequent billing-related processing.

13. The system of claim 12, wherein receiving the one or more user defined rules includes the actions further comprising:
generating a user interface including an input section for receiving the one or more user defined rules; and
displaying the user interface at a display device.

14. The system of claim 12, wherein the one or more provider codes include medical billing codes.

15. The system of claim 12, wherein the one or more provider codes identifies a facility reimbursement fact or a professional reimbursement fact.

16. The system of claim 15, wherein the facility reimbursement fact comprises at least one of an International Classification of Diseases (ICD) code, a Current Procedural Technology (CPT) code, or a Healthcare Common Procedural Coding System Code (HCPCS).

17. The system of claim 15, wherein the professional reimbursement fact comprises at least one of an International Classification of Diseases (ICD) code, a Current Procedural Technology (CPT) code, or a Physician Quality Reporting System (PQRS) code.

18. The system of claim 15, wherein the facility reimbursement fact and the professional reimbursement fact include at least one of a provider name, a provider specialty, a product provided, and a service provided.

19. The system of claim 18, wherein the actions further comprise:
selecting, at random, one of the plurality of patient encounter data sets; and
transmitting the one of the plurality of patient encounter data sets to a documentation specialist for verification.

20. The system of claim 19, wherein selecting the one of the plurality of patient encounter data sets includes further actions comprising selecting a predefined percentage of the plurality of patient encounter data sets.

21-24. (canceled)
